(19) United States
(12) Patent Application Publication
(10) Pub. No.: US 2022/0111102 A1
(43) Pub. Date: Apr. 14, 2022

(54) Capsule for an aromatherapy composition and diffusion device for use with such a capsule

(71) Applicant: Sébastien Fauconnier, Neuvy Saint Se-Pulchre (FR)

(72) Inventor: Sébastien Fauconnier, Neuvy Saint Se-Pulchre (FR)

(21) Appl. No.: 17/420,554

(22) PCT Filed: Dec. 16, 2019
(86) PCT No.: PCT/FR2019/053092

§ 371 Date: Jul. 2, 2021

(30) Foreign Application Priority Data
Jan. 4, 2019 (FR) 1900091

(51) Int. Cl. A61L 9/12

(52) U.S. Cl. CPC A61L 9/12; C11B 9/00; A61L 2209/111; A61L 2209/133

(57) ABSTRACT

A disposable capsule for aerial diffusion of an aromatherapy composition formed from a mixture of essential oil and water, by use of an aerial diffusion device. The capsule includes a shell, the shell having at least a first reservoir containing an essential oil, and a second reservoir containing water, the first reservoir and the second reservoir being hermetically separated from one another.

### CROSS-REFERENCE TO RELATED APPLICATIONS

This application is a Section 371 National Stage Application of International Application No. PCT/FR2019/053092, filed Dec. 16, 2019, the content of which is incorporated herein by reference in its entirety, and published as WO 2020/141268 on Jul. 9, 2020, not in English.

### FIELD OF THE DISCLOSURE

The field of the invention is that of the design and manufacture of devices for the aerial diffusion of an aromatherapy composition, as well as the containers of said aromatherapy compositions.

More specifically, the invention relates to an aerial diffusion device and a disposable capsule comprising an aromatherapy composition, intended to be used in the aerial diffusion device.

### BACKGROUND OF THE DISCLOSURE

In the field of the invention, devices for the aerial diffusion of an aromatherapy composition comprising water and essential oils are known.

Such a device can comprise a base with a receptacle into which the user must pour water and a few drops of essential oils.

By activating the device, a misting system allows water droplets loaded with essential oils to be suspended in the air.

For diffusion, the user must first set up the device by adding water and essential oils. Such an operation allows the user to adjust the aerial diffusion of the aromatherapy composition (choice of essential oils, oil mixtures, etc.) to a certain extent, however it requires a certain knowledge of the essential oils, as well as a certain amount of set up time in order to set up the diffusion device.

Devices for the diffusion of aromatherapy compositions implementing disposable capsules are also known.

These disposable capsules are simply inserted into the delivery and/or diffusion devices. The use of disposable capsules, for example single-dose capsules, is particularly easy to implement for both experienced and novice users.

These disposable capsules can take different shapes and form a reservoir of essential oils.

According to this technique, the disposable capsules require the use of an additional water reservoir. This additional reservoir takes the form of a tank coupled to the machine. It goes without saying that the tank must be refilled periodically.

However, the use of such a water reservoir complicates the use of the diffusion device, which is supposed to be simple thanks to the use of a capsule.

Moreover, when the aerial diffusion of the aromatherapy composition is complete, water is often left in the reservoir. The water reservoir must thus be emptied in order to move the diffusion device without the risk of losing water, even if the reservoir accidentally becomes separated from the diffusion device.

In any case, the water should be emptied from the reservoir to prevent it from becoming stagnant.

Devices have also been developed that allow essential oils to be delivered in the shower. For this purpose, capsules containing essential oils are inserted into a device that is mounted on a water circuit inside the shower, for example.

Finally, disposable capsules comprising essential oils and a specific diffusion medium that does not require water are also known.

Such a capsule complements a diffusion device that is designed so as not to require the use of a water reserve that must be filled. The essential oils are diffused at a low temperature and the capsule forms a self-sufficient consumable.

This last capsule, which is particularly easy to use, does not, however, provide the same experience as that offered by essential oil diffusers of conventional design.

### SUMMARY

The purpose of the invention is in particular to overcome the drawbacks of the prior art. More specifically, the invention aims to provide a disposable capsule containing all of the components of an aromatherapy composition, that is simple to use while allowing for optimised aerial diffusion of the aromatherapy composition.

The invention further aims to provide such a capsule that does not require other elements to be implemented, such as an additional water reservoir of a diffusion device, which needs to be filled in order to carry out aerial diffusion.

The invention further aims to provide a device for the aerial diffusion of an aromatherapy composition which allows the disposable capsules according to the invention to be used in a safe and simple manner for the user.

These objectives, as well as others which will appear hereinbelow, are achieved by means of the invention, which relates to a disposable capsule for aerial diffusion of an aromatherapy composition formed from a mixture of essential oil and water, by means of an aerial diffusion device, comprising a shell, the shell having at least a first reservoir containing an essential oil, characterised in that the shell comprises a second reservoir containing water, the first reservoir and the second reservoir being hermetically separated from one another, and in that it comprises a lid covering the shell, the first reservoir and the second reservoir each being defined between the shell and the lid.

Such a disposable capsule allows different aromatherapy compositions to be proposed, each with different properties. More specifically, each first reservoir of each capsule can contain a different essential oil.

However, at present, special authorisation is required in order to market a mixture of essential oil mixed with another product, for example water. With the disposable capsule according to the invention, this authorisation is no longer necessary since the essential oil and the water are each contained in a separate reservoir. In other words, the essential oil is not mixed with any other product and thus does not require special marketing authorisation.

In particular, the lid facilitates the filling of the capsule prior to marketing. More specifically, the shell can be filled with product, i.e. essential oil and water in each of its reservoirs, and then moved to a new station wherein the lid is applied to the shell to hermetically separate the first reservoir from the second reservoir. Thus, the filling of the disposable capsule can be carried out flat.

Moreover, such a composition of the disposable capsule makes it particularly light.

Advantageously, the disposable capsule comprises:
a first draw-off well communicating with the first reservoir;
a second draw-off well communicating with the second reservoir;
an injection well communicating with the first reservoir.

This ensures that no product leaks out when the water and essential oil are drawn off. More specifically, the wells can be sized such that only a small part of the liquid to be drawn off is present therein, thus avoiding excessive pressure and the risk of product leakage.

Moreover, this creates areas of weakness in the disposable capsule, in this case in the area of the wells from which product can be drawn off while maintaining a high degree of stiffness for the rest of the capsule.

In such a case, the second draw-off well preferably extends beneath the second reservoir towards the first reservoir, in a position of use of the disposable capsule and, the second reservoir has a bottom wall that is inclined relative to the second draw-off well in order to guide the water from the second reservoir towards the second draw-off well.

The geometrical configurations of the second reservoir and of the second draw-off well allow the water contained in the second reservoir to be directed towards the second draw-off well in order to facilitate the drawing off thereof.

This also prevents product from remaining in the disposable capsule at the end of use thereof.

The invention further relates to a device for aerial diffusion of an aromatherapy composition, comprising:
a recess for receiving a capsule as described hereinabove, the capsule comprising a first reservoir containing an essential oil and a second reservoir containing water;
means for diffusing the aromatherapy composition;
means for suctioning the aromatherapy composition out of the capsule,
   characterised in that the device further comprises:
a first needle for drawing water from the second reservoir of the capsule;
a needle for injecting the water drawn from the second reservoir to create the aromatherapy composition by mixing the water and the essential oil in the first reservoir;
a second needle for drawing off the aromatherapy composition coupled to a circuit communicating with the diffusion means;
a pipe connecting the first draw-off needle to the injection needle, the suction means being coupled to the pipe to allow water to be drawn from the second reservoir for injection into the first reservoir.

This aerial diffusion device allows capsules as described hereinabove to be used without the need for them to receive or meet a marketing authorisation requirement.

Furthermore, this device allows, prior to the diffusion of the aromatherapy composition, the water and the oil to be drawn off in order to be mixed and then diffused by the diffusion means.

Advantageously, the device comprises:
a frame carrying the first draw-off needle, the second draw-off needle and the injection needle;
motor means integral with a mount of the device are coupled to each of the first draw-off needle, the second draw-off needle and the injection needle to allow for the displacement thereof relative to the frame between a disengaged position wherein the first draw-off needle, the second draw-off needle and the injection needle are spaced apart from the receiving recess, and an active position wherein the first draw-off needle, the second draw-off needle and the injection needle are inserted into the receiving recess.

Thus, thanks to the motor means, the risks of unintentional piercing of the capsule are eliminated, or almost eliminated.

Moreover, thanks to their disengaged position, there is no risk of the needles damaging the capsule while it is being inserted into the aerial diffusion device. This prevents the one or more liquids contained in the capsule from leaking inside the aerial diffusion device.

Preferably, the aerial diffusion device comprises transmission means inserted between the motor means and the needles to allow the needles to be simultaneously displaced from the disengaged position thereof to the active position thereof or vice-versa.

This in particular facilitates the displacement of each of the needles such that the capsule is pierced at several points simultaneously. Simultaneously piercing the capsule at several points prevents a first opening from being made in the capsule such that the liquid is suctioned without being able to be injected or used directly if another needle is not connected to the capsule or directed towards the diffusion means.

In other words, any uncontrolled piercing of the disposable capsule, which could cause product leakage (leakage of water and/or essential oil) inside the machine, is prevented, such leakage potentially hindering the correct performance of the aerial diffusion device. According to one preferred embodiment, the transmission means comprise:
a driving pinion coupled to the motor means;
a plurality of follower gears, each follower gear being associated with one of the first draw-off needle, the second draw-off needle and the injection needle;
an idler gear meshing with each of the follower gears,
   the driving pinion driving one of the follower gears in rotation, which in turn drives the idler gear, the idler gear then transmitting the rotational motion thereof to the other follower gears such that all of the follower gears rotate in the same direction of rotation.

Such transmission means are both simple to implement and safe for the operation of the aerial diffusion device. More specifically, the use of pinions and follower gears prevents the pipes associated with the needles from twisting. More specifically, by rotating in the same direction of rotation, each follower gear allows the needles to be displaced into their position without resulting in a twist in a first direction at a first end of a pipe and a twist in a second direction at a second end of the same pipe, these two twists in opposite directions being capable of weakening or even damaging the pipe in the long term.

Advantageously, the frame has stop means for stopping the rotation of the needles, each needle being integral with a follower gear via a helical connection, the stop means transforming the rotational motion of the needles into a translational motion of the needles from the disengaged position thereof to the active position thereof or vice-versa. The stop means thus enable the rotational motion of the follower gears to be transformed into a translational motion of the needles. In this way, the needles act on the capsule by piercing it without causing a rotational motion that could damage the capsule and create pathways allowing the fluid to escape from the capsule.

In other words, when the needle is inserted into the capsule, the walls of the shell are pierced by the thrust of the needle alone. A rotational motion could instead create a tearing of the shell material, creating fluid pathways and thus product leakage inside the aerial diffusion device.

According to one specific embodiment, the aerial diffusion device further comprises a needle allowing air to penetrate inside the second reservoir, the air penetration needle being capable of moving relative to the frame between a disengaged position wherein it is spaced apart from the receiving recess, and an active position wherein it is inserted into the receiving recess.

Such a vent facilitates water draw-off since air can penetrate the second reservoir such that the suction means do not have to create a significant negative pressure. More generally, it simplifies the design of the machine and makes it less energy consuming.

### BRIEF DESCRIPTION OF THE DRAWINGS

Other features and advantages of the invention will be better understood upon reading the following description given of a non-limiting, preferred embodiment of the invention, provided for illustration purposes, and with reference to the accompanying drawings, in which:

FIG. 1 is a front view of a disposable capsule for aerial diffusion of an aromatherapy composition, according to the invention;

FIG. 2 is a side view of the disposable capsule according to the invention;

FIG. 3 is a perspective view of the disposable capsule according to the invention;

FIG. 4 is a detailed view of a device for aerial diffusion of an aromatherapy composition, according to the invention, wherein a disposable capsule according to the invention is inserted;

FIG. 5 is a perspective, rear view of a mechanism for drawing off an aromatherapy composition of the diffusion device according to the invention;

FIG. 6 is a perspective, front view of the mechanism for drawing off an aromatherapy composition of the diffusion device according to the invention;

FIG. 7 is a detailed, back view showing meshing of the mechanism for drawing off an aromatherapy composition of the diffusion device according to the invention;

FIG. 8 is a sectional view of the diffusion device according to the invention, showing means for diffusing the aromatherapy composition.

### DETAILED DESCRIPTION OF ILLUSTRATIVE EMBODIMENTS

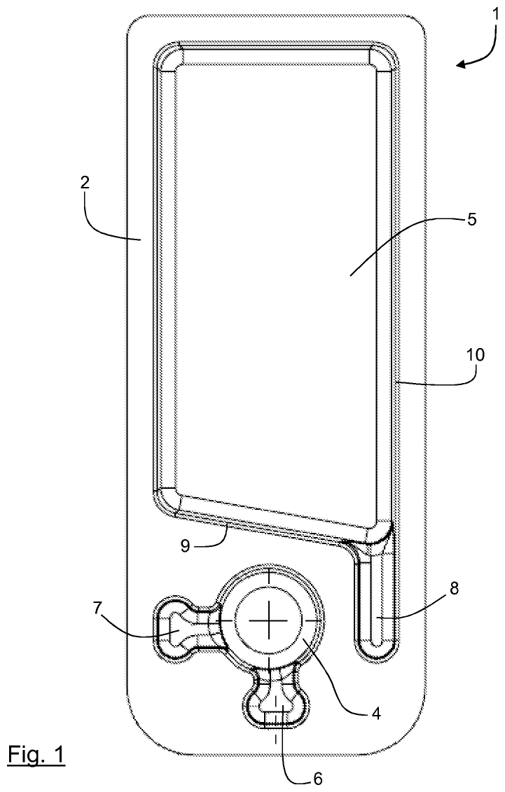

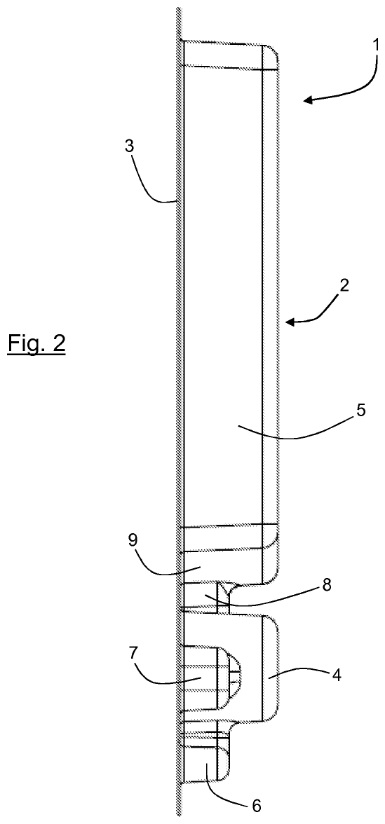

FIGS. 1 to 3 show a disposable capsule 1 according to the invention, intended to contain an aromatherapy composition.

The disposable capsule 1 comprises a shell 2 and a lid 3 intended to cover and close the shell 2.

More specifically, the shell 2 has a first reservoir 4 and a second reservoir 5.

The first reservoir 4 is intended to contain an essential oil and the second reservoir 5 is intended to contain water.

When the water and the essential oil are mixed, they jointly form the aromatherapy composition as described hereinbelow.

The first reservoir 4 and the second reservoir 5 are disposed one above the other in a position of use of the capsule 1.

For information, the first reservoir 4 has a capacity comprised between 1.6 ml and 2 ml, and the second reservoir 5 has a capacity comprised between 15 ml and 20 ml.

More specifically, the first reservoir 4 is located in a lower part of the capsule 1, whereas the second reservoir 5 is located in an upper part of the capsule 1.

The first reservoir 4, which is intended to contain the essential oil, has a substantially cylindrical shape.

A first draw-off well 6 and an injection well 7 extend from the substantially cylindrical shape.

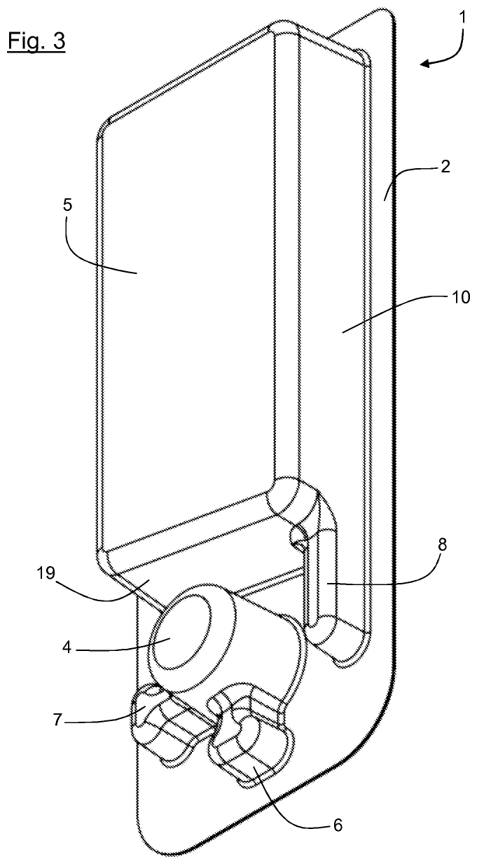

More specifically, both the first draw-off well 6 and the injection well 7 are substantially T-shaped with the web extending from the cylindrical part of the first reservoir 4 towards the outside of the capsule 1 and the flange extending substantially perpendicular to the web. As shown in FIGS. 1 and 3 in particular, the first draw-off well 6 extends downwards from the first reservoir 4 and the injection well 7 extends perpendicularly to the extension of the first draw-off well 6.

In other words, the first draw-off well 6 extends in a first direction that is coincident with the alignment of the first reservoir 4 and of the second reservoir 5.

The T-shape of the injection well 7 and of the first draw-off well 6 creates a reinforced contour of the wells 6, 7 to ensure the safe draw-off of the water or of the aromatherapy composition, as explained hereinbelow.

The second reservoir 5 has a substantially parallelepiped shape and comprises a second draw-off well 8.

The second draw-off well 8 extends from the second reservoir 5 towards the first reservoir 4.

More particularly, as shown in FIG. 1, the second draw-off well 8 extends from a corner of the second reservoir 5 located in a low portion of the second reservoir 5.

A bottom wall 9 of the second reservoir 5, i.e. a wall facing the first reservoir 4, has an inclination such that this bottom wall forms, together with a side wall 10, a funnel for directing the water contained in the second reservoir 5 towards the second draw-off well 8.

The second draw-off well 8 extends substantially vertically and has a reinforced rim to ensure that water is safely drawn from the second well 8.

Preferably, the shell 2 is made of a plastic material such as PET, particularly known for the manufacture of food containers, and the lid 3 is made of an aluminium foil.

The assembly of the lid 3 on the shell 2 allows the lid 3 to be welded to the shell 2 around the peripheries of each of the reservoirs 4, 5. Thus, the first reservoir 4 and the second reservoir 5 are hermetically separated from one another.

In other words, when the capsule 1 is assembled, i.e. when the lid 3 is sealed by welding on the shell 2, the product contained in the first reservoir 4 cannot mix with the product contained in the second reservoir 5.

In other words, when the capsule 1 is assembled, the water and the essential oil cannot be mixed unless the capsule 1 is damaged.

Such a capsule 1 is particularly advantageous because, in cosmetic products, of which aromatherapy compositions are a part, when an essential oil is mixed with another substance, such as water or another liquid or semi-liquid product, marketing authorisation must be obtained in order to ensure that the mixture of the essential oil with the other product does not present any health risk to a user.

Thanks to this capsule, the essential oil is isolated from the water, so there is no need to obtain such authorisation.

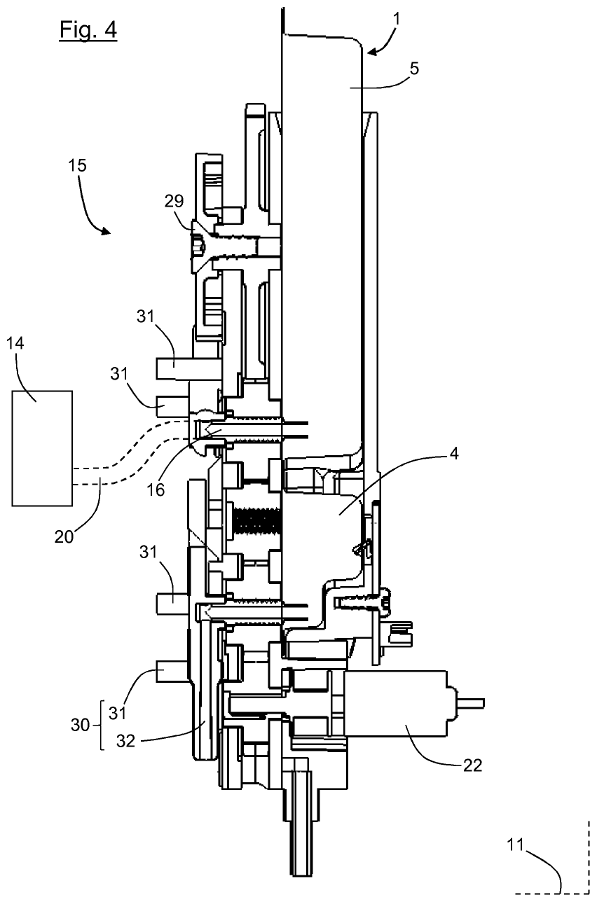

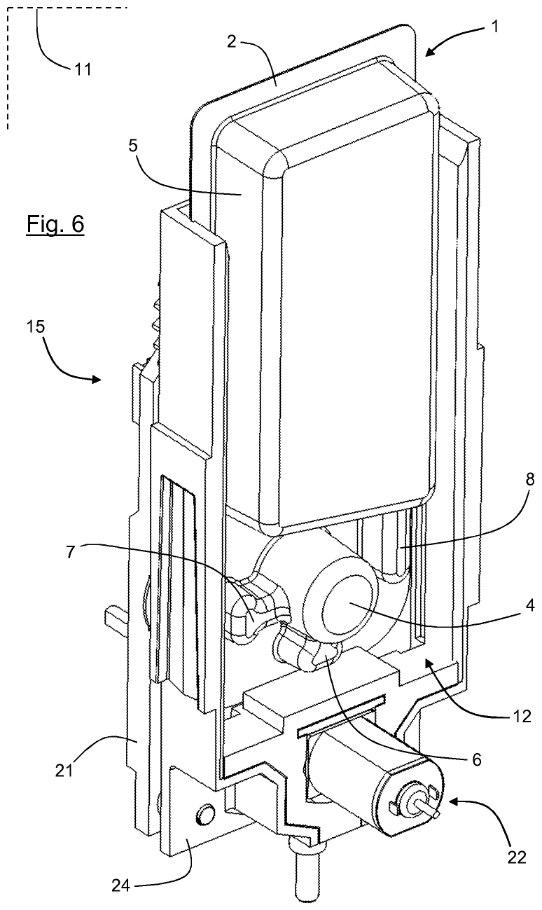

A device 11 for aerial diffusion of an aromatherapy composition is described with reference to FIGS. 4 to 8. According to the invention, this aerial diffusion device 11 comprises:
a recess 12 for receiving a disposable capsule 1 as previously described;
means 13 for diffusing the aromatherapy composition;
means 14 for suctioning the aromatherapy composition out of the capsule 1 for the diffusion thereof by the diffusion means 13.

The device 11 further comprises a unit 15 for drawing off the aromatherapy composition incorporating:
a first needle 16 for drawing water from the second reservoir 5 of the capsule 1;
a needle 17 for injecting the water drawn into the first reservoir 4 to create the aromatherapy composition by mixing the water and the essential oil inside the first reservoir 4;
a second needle 18 for drawing off the aromatherapy composition coupled to a circuit 19 communicating with the diffusion means 13.

The first draw-off needle 16 is intended, as shown in FIG. 4 in particular, to penetrate the second reservoir 5, and more particularly the second draw-off well 8.

The injection needle 17 is intended to pierce the capsule 1 to penetrate the injection well 7 of the first reservoir 4.

The second draw-off needle 18 is intended to penetrate the first draw-off well 6 of the first reservoir 4 such that the aromatherapy composition contained therein can be drawn off for diffusion by the diffusion means 13.

The diffusion device 11 further comprises at least one pipe 20 connecting the first draw-off needle 16 to the injection needle 17 so that the water drawn from the second reservoir 5 can be injected into the first reservoir 4.

In order to draw off the water and then inject it into the first reservoir 4, the diffusion device 11 uses suction means 14, which in particular take the form of a peristaltic pump. This peristaltic pump is in particular coupled to the pipe 20 connecting the first draw-off needle 16 to the injection needle 17, so as to draw water from the second reservoir 5 for injection into the first reservoir 4.

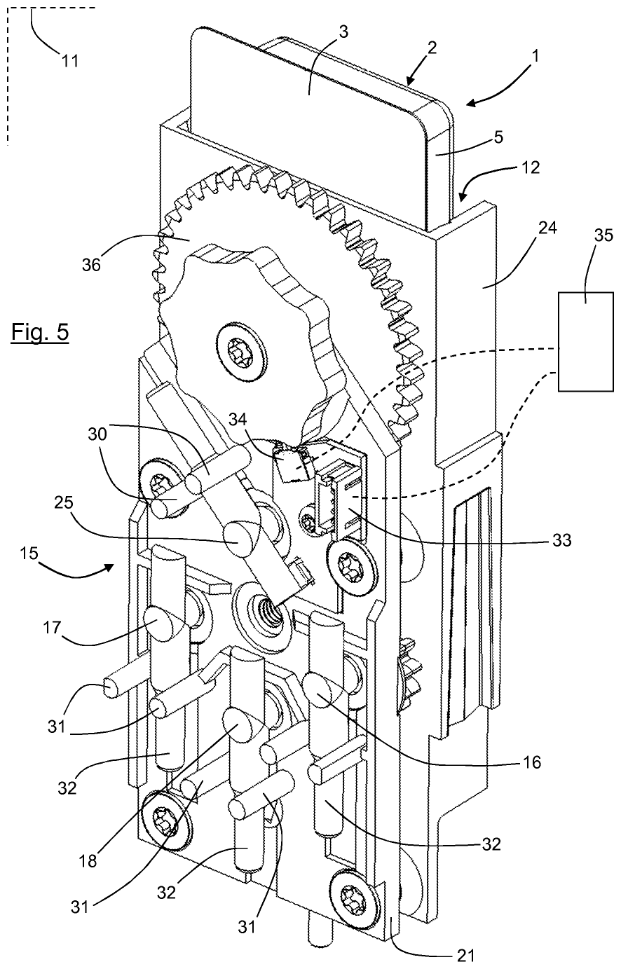

The aerial diffusion device 11 comprises, as shown in FIGS. 5 to 7, a frame 21, motor means 22 and transmission means 23.

More specifically, the frame 21 is mounted on a mount 24 defining the recess 12 for receiving the capsule 1, and carries the first draw-off needle 16, the second draw-off needle 18 and the injection needle 17.

The motor means 22 are integral with the mount 24 and are coupled to each of the first draw-off needle 16, the second draw-off needle 18 and the injection needle 17 in order to enable the displacement thereof to draw the water and the aromatherapy composition from the disposable capsule 1.

More specifically, each of the first draw-off needle 16, the second draw-off needle 18 and the injection needle 17 are capable of moving relative to the frame 21 between a disengaged position wherein the first draw-off needle 16, the second draw-off needle 18 and the injection needle 17 are spaced apart from the recess 12 for receiving the disposable capsule 1, and thus from the capsule 1 itself, and an active position wherein the first draw-off needle 16, the second draw-off needle 18 and the injection needle 17 are inserted into the recess 12 for receiving the capsule 1, and thus into the capsule 1 itself.

In the disengaged position of the needles 16, 17, 18, the recess 12 is freely accessible such that the capsule 1 can be inserted therein or removed therefrom without risk of being scratched or pierced by the needles 16, 17, 18.

Furthermore, in order to ensure that water is easily drawn from the second reservoir 5, the aerial diffusion device 11 further comprises an air penetration needle 25 for allowing air to penetrate the second reservoir 5.

More specifically, this air penetration needle 25 forms a vent allowing air to enter the second reservoir 5 naturally when the water contained in the second reservoir 5 is removed via the first draw-off needle 16.

This in particular prevents the suction means 14 from being oversized and/or water from remaining trapped inside the second reservoir 5.

Furthermore, the presence of the vent prevents the capsule 1 from needing to be deformed when the water is drawn from the second reservoir 5.

Similarly to the first draw-off needle 16, the second draw-off needle 18 and the injection needle 17, the air penetration needle 25 is mounted on the frame 21 and is capable of moving relative thereto between a disengaged position wherein the air penetration needle 25 is spaced apart from the recess 12 for receiving the disposable capsule 1, and thus from the capsule 1 itself, and an active position wherein the air penetration needle 25 is inserted into the recess 12 for receiving the capsule 1, and more specifically into the second reservoir 5.

The transmission means 23 comprise:
a driving gear 26 integral with the motor means 22;
follower gears 27 integral with each of the needles 16, 17, 18, 25;
an idler gear 28 meshing with each of the follower gears 27.

More specifically, each needle 16, 17, 18, 25 is integral with one of the follower gears 27 by means of a helical connection.

As shown in FIG. 4, each needle 16, 17, 18, 25 is integral with a screw 29 mounted in a follower gear 27 by a helical connection. Each needle 16, 17, 18, 25 thus passes through the screw 29 in which it is embedded.

The frame 21 further includes stop means 30 intended to prevent the needles 16, 17, 18, 25 from rotating when the follower gears 27 are driven in rotation.

More specifically, the stop means 30 comprise pins 31 integral with the frame 21 and splines 32 each intended to receive one of the needles 16, 17, 18, 25.

The splines 32 are inserted between two pins 31 such that they prevent the rotation thereof.

Thus, during operation of the transmission means 23, the rotational motion of the follower gears 27 is transformed into a translational motion of the needles 16, 17, 18, 25 by the helical connection between the needle 16, 17, 18, 25, and more particularly the screw 29 with which it is integral, and the follower gear 27.

The translational motion is due in particular to the presence of the stop means 30, which prevent the rotation of the needles 16, 17, 18, 25 and, via the helical connection, transform the rotational motion of the follower gears 27 into a translational motion of the needles 16, 17, 18, 25 from the disengaged position thereof to the active position thereof, or vice-versa.

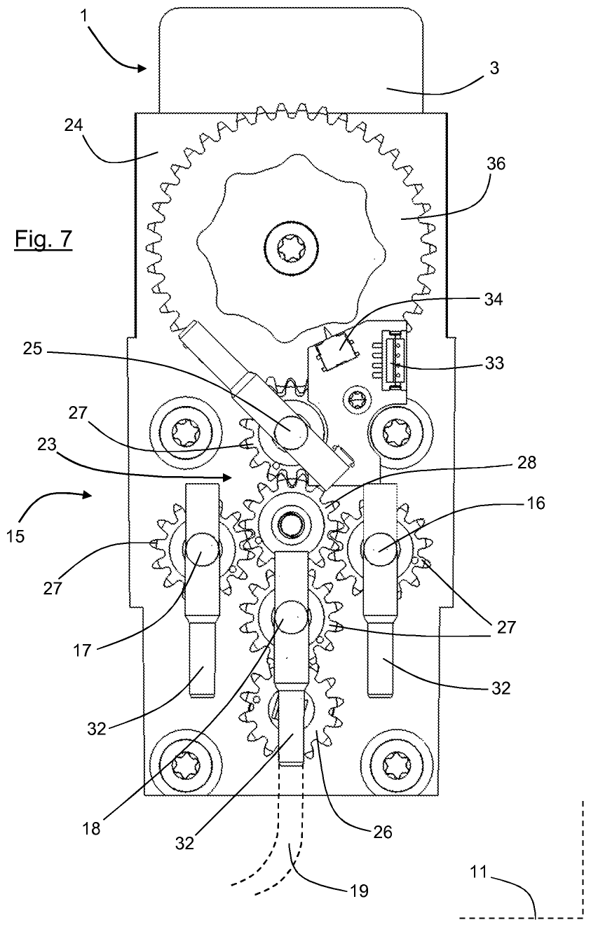

With reference to FIG. 7, the driving gear 26 meshes with one of the follower gears 27, i.e. the follower gear 27 which is integral with the second draw-off needle 18, which in turn meshes with the idler gear 28.

The direction of rotation of the follower gear 27 driven by the driving gear 26 is maintained and transmitted to the other follower gears 27 by the meshing of the idler pinion 28 with each of the other follower gears 27.

This creates a simultaneous translational motion of each of the needles 16, 17, 18, 25, from the disengaged position thereof to the active position thereof, or vice-versa.

In other words, when the motor means 22 are activated, all of the needles 16, 17, 18, 25 are displaced simultaneously. The displacement of the needles 16, 17, 18, 25 is thus synchronised.

Thus, when the first draw-off needle 16 pierces the shell 2 of the disposable capsule 1, the other needles 17, 18, 25 also pierce the shell 3 of the capsule 1.

Still with reference to FIG. 7, the diffusion device 11 comprises a first sensor 33 intended to detect a predetermined penetration of the needles 16, 17, 18, 25 into the capsule 1, and a second sensor 34 intended to detect the withdrawal of the needles 16, 17, 18, 25 relative to the capsule 1.

More particularly, the first sensor 33 is intended to monitor and inform the driving means 22 that the needles 16, 17, 18, 25 are in the active position thereof, and the second sensor is intended to inform the driving means 22 that the needles 16, 17, 18, 25 are in the disengaged position thereof.

This in particular allows the displacement of the needles 16, 17, 18, 25 between the two positions thereof to be limited automatically.

In particular, the sensors 33, 34 are associated with a control unit 35 of the diffusion device 11, comprising a processing unit and a chronometer or a clock for defining a duration of use of the diffusion device 11.

More specifically, the control unit 35 is informed of the active position of the needles 16, 17, 18, 25 and then triggers a time delay allowing the needles 16, 17, 18, 25 to be held in the active position thereof until the end of the time delay corresponding to the use of the entire aromatherapy composition.

When the time delay has elapsed, the control unit 35 drives the motor means 22 to position the needles 16, 17, 18, 25 into the disengaged position thereof and thus allow the capsule 1 to be removed from the receiving recess 12.

Manual control means can also be added to the aerial diffusion device 11 to allow the needles 16, 17, 18, 25 to be displaced manually into the disengaged position thereof when the time delay has not fully elapsed.

The second sensor 34 is in particular connected to a free wheel 36 which is integral with one of the follower gears 27, this free wheel 36 having a marking intended to inform the sensor of the position of the needles 16, 17, 18, 19.

The first sensor 33 can be a touch sensor, i.e. a pressure sensor intended to detect contact of a needle 16, 17, 18, 25 (or of the spline 32 associated therewith) against the first sensor 33. When the first sensor 33 detects this contact, it deduces that the needles 16, 17, 18, 25 are in the active position thereof.

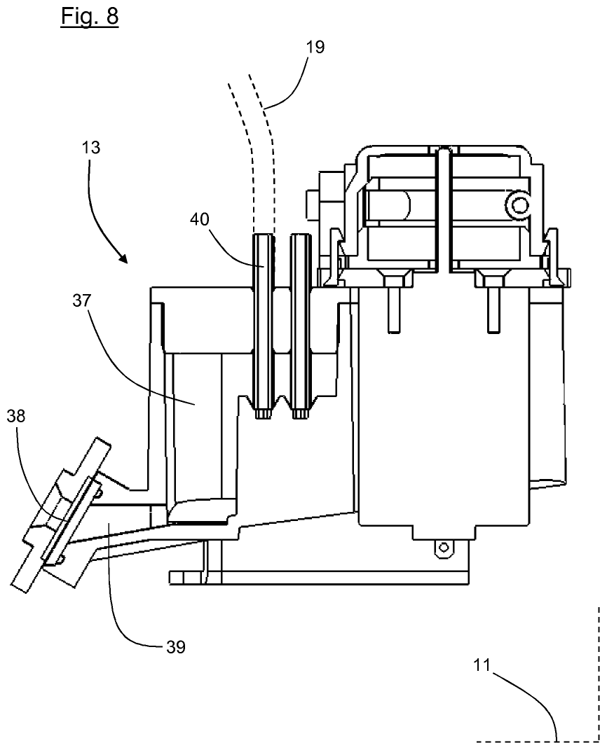

With reference to FIG. 8, the diffusion means 13 comprise a reservoir 37, a vibrating diaphragm 38, and a duct 39 connecting the reservoir 37 to the vibrating diaphragm 38. The reservoir 37 has a tubular inlet 40 connected to the circuit 19 such that when the aromatherapy composition is in the reservoir 37, it can only be directed towards the vibrating diaphragm 38.

Thus, even if the device 11 is positioned upside down, the aromatherapy composition cannot leak from the reservoir 37.

The vibrating diaphragm is advantageously controlled by the receipt of the aromatherapy composition. In other words, in the absence of any aromatherapy composition, the vibrating diaphragm 38 is not activated.

During use, when the first draw-off needle 16 pierces the shell 2 of the capsule 1, the other needles 17, 18, 25 also pierce the shell 2 of the capsule 1.

More specifically:
the first draw-off needle 16 pierces the shell 2 at the second draw-off well 8;
the injection needle 17 pierces the shell 2 at the injection well 7;
the second draw-off needle 18 pierces the shell 2 at the first draw-off well 6; and
the air penetration needle 25 pierces the shell 2 directly in the second reservoir 5.

When the needles 16, 17, 18, 25 are in the active position thereof, the suction means 14 are actuated such that the water contained in the second reservoir 5 is suctioned by the first draw-off needle 16, passes through the pipe 20 to reach the injection needle 17 and is injected into the injection well 7 of the first reservoir 4.

In the first reservoir 4, the water is mixed with the oil to form the aromatherapy composition, and is then drawn from the first draw-off well 6 by the second draw-off needle 18.

The aromatherapy composition then reaches the diffusion means 13 via the circuit 19.

The aromatherapy composition then flows from the reservoir 37 to the vibrating diaphragm 38 via the duct 39. On contact with the aromatherapy composition, the vibrating diaphragm 38 then vibrates in order to mist the aromatherapy composition.

Although the present disclosure has been described with reference to one or more examples, workers skilled in the art will recognize that changes may be made in form and detail without departing from the scope of the disclosure and/or the appended claims.

1. A disposable capsule for aerial diffusion of an aromatherapy composition formed from a mixture of essential oil and water, by using of an aerial diffusion device, the disposable capsule comprising:
a shell, the shell having at least a first reservoir containing an essential oil, and a second reservoir containing water, the first reservoir and the second reservoir being hermetically separated from one another; and
a lid covering the shell, the first reservoir and the second reservoir each being defined between the shell and the lid.

2. The disposable capsule according to claim 1, which comprises:
a first draw-off well communicating with the first reservoir;
a second draw-off well communicating with the second reservoir; and
an injection well communicating with the first reservoir.

3. The disposable capsule according to claim 2, wherein the second draw-off well extends beneath the second reservoir towards the first reservoir, in a position of use of the disposable capsule, and the second reservoir has a bottom wall that is inclined relative to the second draw-off well in order to guide the water from the second reservoir towards the second draw-off well.

4. An aerial diffusion device for aerial diffusion of an aromatherapy composition, comprising:
a recess for receiving a capsule comprising a first reservoir containing an essential oil and a second reservoir containing water;
a diffuser for diffusing the aromatherapy composition;
a suction device for suctioning the aromatherapy composition out of the capsule;
a first draw-off needle for drawing water from the second reservoir of the capsule;
an injection needle for injecting the water drawn into the first reservoir to create the aromatherapy composition by mixing the water and the essential oil in the first reservoir;
a second draw-off needle drawing off the aromatherapy composition coupled to a circuit communicating with the diffuser; and
a pipe connecting the first draw-off needle to the injection needle, the suction device being coupled to the pipe to allow water to be drawn from the second reservoir for injection into the first reservoir.

5. The aerial diffusion device according to claim 4, which further comprises:
a frame carrying the first draw-off needle, the second draw-off needle and the injection needle; and
a motor integral with a mount of the device and coupled to each of the first draw-off needle, the second draw-off needle and the injection needle to allow for the displacement thereof relative to the frame between a disengaged position wherein the first draw-off needle, the second draw-off needle and the injection needle are spaced apart from the receiving recess, and an active position wherein the first draw-off needle, the second draw-off needle and the injection needle are inserted into the receiving recess.

6. The aerial diffusion device according to claim 5, which further comprises a transmitter inserted between the motor and the first draw-off needle, the second draw-off needle and the injection needle to allow the first draw-off needle, the second draw-off needle and the injection needle to be simultaneously displaced from the disengaged position thereof to the active position thereof, or vice-versa.

7. The aerial diffusion device according to claim 6, wherein the transmitter comprises:
a driving pinion coupled to the motor;
a plurality of follower gears, each follower gear being associated with one of the first draw-off needle, the second draw-off needle and the injection needle; and
an idler gear meshing with each of the follower gears;
the driving pinion driving one of the follower gears in rotation, which in turn drives the idler gear, the idler gear then transmitting the rotational motion thereof to the other follower gears such that all of the follower gears rotate in the same direction of rotation.

8. The aerial diffusion device according to claim 7, wherein the frame has a stop for stopping the rotation of the needles, and each needle is integral with the associated follower gear via a helical connection, the stop transforming the rotational motion of the needles into a translational motion of the needles from the disengaged position thereof to the active position thereof or vice-versa.

9. The aerial diffusion device according to claim 4, which further comprises an air penetration needle allowing air to penetrate inside the second reservoir, the air penetration needle being capable of moving relative to the frame between a disengaged position wherein the air penetration needle is spaced apart from the receiving recess, and an active position wherein the air penetration needle is inserted into the receiving recess.
